(19) United States
(12) Patent Application Publication
(10) Pub. No.: US 2026/0196309 A1
(43) Pub. Date: Jul. 9, 2026

(54) DETERMINING PHYSICAL PROPERTIES OF ALKALI-ACTIVATED CONCRETE

(71) Applicant: UNIVERSITEIT GENT, Gent (BE)

(72) Inventors: Beibei SUN, Guangdong (CN); Luchuan DING, Shanghai (CN); Guang YE, Den Haag (NL); Geert DE SCHUTTER, Gent (BE)

(21) Appl. No.: 19/131,943

(22) PCT Filed: Nov. 22, 2023
(86) PCT No.: PCT/EP2023/082679

§ 371 Date: May 21, 2025

(30) Foreign Application Priority Data
Nov. 23, 2022 (EP) 22208981.5

(51) Int. Cl. G16C 20/30; G16C 20/70; G16C 60/00

(52) U.S. Cl. CPC G16C 20/30; G16C 20/70; G16C 60/00

(57) ABSTRACT

A computer-implemented method is provided (200) for determining one or more physical properties of alkali activated concrete, AAC, from a mix proportion (201) of an AAC mixture. The AAC mixture has one or more precursors, one or more chemical compounds, and water according to the mix proportion. The computer implemented method includes obtaining (210) one or more control factors (211-215) from the mix proportion of the AAC mixture, where the one or more control factors are indicative for one or more reaction mechanisms in the AAC mixture; and determining (220) a compressive strength (225) of the AAC based on the one or more control factors of the AAC mixture by means of a machine learning model (224). The machine learning model is trained by a training dataset (221) having one or more control factors of a plurality of AAC mixtures with different mix proportions; and corresponding compressive strengths of the AAC obtained by setting and hardening of the respective AAC mixtures.

### FIELD OF THE INVENTION

The present invention generally relates to predicting physical properties of alkali-activated concrete.

### BACKGROUND OF THE INVENTION

Alkali-activated concrete, AAC, is an environmental friendly alternative to traditional concrete, e.g. Portland cement concrete, which typically requires substantial amounts of natural resources and emits substantial amounts of carbon dioxide during its production process. AAC typically comprises one or more precursors rich in alumina and/or silica, an alkaline activator to initiate the setting and hardening reactions, one or more aggregates, and water. The precursors are typically industrial by-products, e.g. fly ash and/or blast furnace slag, further contributing to the ecological appeal of AAC.

The ratio of these constituents in AAC, i.e. the AAC mix proportion, determine the mechanical properties and durability of the cured concrete structure. It is a problem to predict these mechanical properties of AAC based on the AAC mix proportion. Typically, a trial-and-error approach is adopted wherein a candidate mix proportion is produced and a sample is cured for several days, e.g. 28 days, before experimentally testing the physical properties, e.g. compressive strength. It can thus be desirable to predict the physical properties of AAC based on the mix proportion of an AAC mixture to avoid or limit the time and labour for testing the candidate mix proportion.

To this end, some models to predict physical properties of AAC have been developed. These models are typically limited to predicting a single physical property for a mix of specific constituent materials, a narrow range of mix proportions, and a predetermined curing condition. Additionally, these models are typically based on a limited database of experimentally determined physical properties, and consider correlated input variables which can lead to over-featuring or over-fitting of the prediction model. As such, the broad applicability of prediction models for predicting physical properties of varying AAC mixtures is a challenge.

### SUMMARY OF THE INVENTION

It is an object of the present invention, amongst others, to solve or alleviate the above identified problems and challenges by improving the prediction of physical properties of AAC.

According to a first aspect, this object is achieved by a computer-implemented method for determining one or more physical properties of alkali activated concrete, AAC, from a mix proportion of an AAC mixture. The AAC mixture comprises a first chemical compound of an alkali activator, a second chemical compound of the alkali activator, a first precursor, a second precursor, and water according to the mix proportion. The computer implemented method comprises obtaining control factors from the mix proportion of the AAC mixture, wherein the control factors are weight ratios including a weight ratio of water to the first and second precursor, a weight ratio of the first precursor to the first and second precursor, a weight ratio of the first chemical compound to the first and second precursor, and a weight ratio of the second chemical compound to the first chemical compound; and determining a compressive strength of the AAC based on the control factors of the AAC mixture by means of a machine learning model. The machine learning model is trained by a training dataset comprising control factors of a plurality of AAC mixtures characterized by different mix proportions; and corresponding compressive strengths of the AAC obtained by setting and hardening of the respective AAC mixtures.

The first and second chemical compounds form an alkali activator that enables the precursors to react, thereby initiating the setting and hardening reaction mechanisms of the AAC mixture. The precursors may be industrial by-products rich in alumina and/or silica. In addition to the precursors, the first and second chemical compounds, and water; the AAC mixture may comprise one or more substantially inert granular materials, i.e. aggregates.

The control factors may be indicative for one or more reaction mechanisms that occur during setting and/or hardening of the AAC mixture due to mixing the constituents of the AAC mixture according to the mix proportion. The control factors may be associated with the mix proportion of the AAC mixture. A control factor may, for example, be indicative for the composition of reaction products, the structure of reaction products, a reaction rate of reactants, a dissolution rate of reactants, a condensation rate of reactants, or a saturation of reactants during or after hardening of the AAC mixture. As such, these control factors are characteristic for the physical properties of the obtained AAC. The control factors may, for example, be relative amounts of the precursors, the chemical compounds, water, or a combination thereof. The control factors are obtained from the mix proportion of the AAC mixture. These control factors allow determining the compressive strength of the AAC without considering the aggregates within the AAC mixture as the aggregates are inert. This has the advantage that the compressive strength can be determined or predicted for a variety of AAC mixtures. In other words, the computer implemented method is broadly applicable to determine the compressive strength of AAC mixtures with substantially varying compositions, e.g. AAC mixtures that include different aggregates. By limiting the number of control factors, the over-featuring of the trained machine learning model can further be avoided.

The training dataset may, for example, be a database of experimentally tested AAC mixtures wherein the control factors of the AAC mixtures are mapped to the respective compressive strengths of the obtained AAC. The training dataset may comprise a substantial number of experimentally tested AAC mixtures, in particular at least around 800 experimentally tested AAC mixtures, preferably more than 800 experimentally tested AAC mixtures. The experimentally tested AAC mixtures in the training dataset may further comprise AAC mixtures with substantially varying mix proportions and/or constituent materials, i.e. precursors and chemical compounds. This has the further advantage that the trained machine learning model can be more accurate and can be more broadly applicable to determine the compressive strength of AAC mixtures with varying compositions. It is a further advantage that the compressive strength can be determined for AAC mixtures comprising a plurality of precursors and/or a plurality of chemical compounds.

The trained machine learning model thus allows determining or predicting the compressive strength of AAC from a predetermined selection of control factors that are obtainable from the mix proportion of the AAC mixture and that are indicative for one or more reaction mechanisms in the AAC mixture. This has the further advantage that the compressive strength of an AAC mixture can be predicted in a cost-efficient manner, as the time and labour to determine the compressive strength of an AAC mixture can be reduced by avoiding curing and testing of AAC mixtures. It is a further advantage that the trained machine learning model can be implemented in a mix design process for designing the mix proportion of an AAC mixture that yields AAC with desired physical properties. It is a further advantage that this can improve the utilization of AAC as a more ecological alternative to traditional concrete.

The control factors are weight ratios indicative for a weight of one or more precursors, one or more chemical compounds, or water within the AAC mixture compared to a weight of one or more precursors, one or more chemical compounds, or water within the AAC mixture.

The control factors may thus, for example, be a ratio between a weight of precursor and a total weight of precursor in the AAC mixture; a ratio between a weight of water and a weight of a chemical compound; a weight ratio between two chemical compounds; or any other weight ratio. This allows obtaining the control factors from the mix proportion of the AAC mixture in a straightforward manner. Alternatively, the control factors may be volume ratios indicative for a volume of one or more precursors, one or more chemical compounds or water within the AAC mixture compared to a volume of one or more precursors, one or more chemical compounds, or water within the AAC mixture.

The AAC mixture comprises a first chemical compound of an alkali activator, a second chemical compound of an alkali activator, a first precursor, and a second precursor.

It is a further advantage that the computer implemented method may be material-agnostic, i.e. that the computer-implemented method may be applied to AAC mixture comprising different precursors and chemical compounds.

The weight ratios include a weight ratio of water to the first and second precursor, a weight ratio of the first precursor to the first and second precursor, a weight ratio of the first chemical compound to the first and second precursor, and a weight ratio of the second chemical compound to the first chemical compound.

This allows determining the compressive strength of the AAC accurately and reliably, as these weight ratios are substantially related to the compressive strength of the AAC. In particular, the weight ratio of the first precursor to the first and second precursor in the AAC mixture may be most closely related to the compressive strength of the AAC. The decreasing order of significance of the control factors may further be the weight ratio of the first chemical compound to the first and second precursor, the weight ratio of the second chemical compound to the first chemical compound, and the weight ratio of water to the first and second precursor. A limited number of control factors has the further advantage that over-featuring of the trained machine learning model is avoided.

According to an embodiment, the first chemical compound is sodium oxide, Na₂O, the second chemical compound is silicon dioxide, SiO₂, the first precursor is blast furnace slag, BFS, and the second precursor is fly ash, FA.

According to an embodiment, determining the compressive strength of the AAC is further based on a curing time and/or a curing condition of the AAC mixture.

The curing time may be indicative for an elapsed time between the moment of initiating the hardening reaction of the AAC mixture, e.g. when placing or pouring the AAC mixture, and the moment when the compressive strength is to be determined, e.g. 28 days after placing the AAC mixture. The curing time may be expressed as a unit of time, e.g. hours or days. The curing condition may be indicative of the environmental conditions during the setting reactions of the AAC mixture, e.g. ambient temperature or humidity.

According to an embodiment, the curing time of the mixture is between about 1 day and about 180 days.

In other words, the trained machine learning model may preferably provide reliable and accurate predictions of the physical properties of AAC after a curing time between around 1 day and around 180 days. The curing time may thus be an input variable provided to the machine learning model, e.g. provided by a user of the machine learning model.

According to an embodiment, the machine learning model may be a gradient boosted regression tree, GBRT, model, a random forest, RF, model, an artificial neural network, ANN, model, or a regression tree, RT, model.

Preferably, the machine learning model may be a gradient boosted regression tree model as this allows more accurate, more efficient, and faster determining of the compressive strength of the AAC.

According to an embodiment, the computer implemented method may further comprise determining a flexural strength, a splitting tensile strength, and/or an elastic modulus of the AAC based on respective predetermined relationships with the determined compressive strength.

Thus, quantitative predetermined relationships, e.g. functions, may be identified that allow determining other physical properties of the AAC from the compressive strength predicted by the machine learning model. In other words, the determined compressive strength may be an input variable for the respective predetermined relationships, which output the predicted flexural strength, splitting tensile strength, and/or elastic modules of the AAC, respectively. This allows predicting a plurality of physical properties of AAC from the mix proportion of the AAC mixture. This has the further advantage that the time and labour to determine physical properties of an AAC mixture can further be reduced by further avoiding the curing and testing of the AAC mixture. The respective predetermined relationships may, for example, be determined based on a dataset of experimentally tested AAC mixtures comprising their respective physical properties.

According to an embodiment, the respective predetermined relationships may be square root relationships between the determined compressive strength and the flexural strength; the determined compressive strength and the splitting tensile strength; and/or the determined compressive strength and the elastic modules.

These predetermined relationships allow determining a plurality of physical properties of the AAC fast and computationally efficient, as the computations for determining the flexural strength, splitting tensile strength, and elastic modulus require only limited computing resources.

According to a second aspect, the object is achieved by a data processing system configured to perform the computer implemented method according to the first aspect.

According to a third aspect, the object is achieved by a computer program comprising instructions which, when the computer program is executed by a computer, cause the computer to perform the computer implemented method according to the first aspect.

According to a fourth aspect, the object is achieved by a computer-readable medium comprising instructions which, when executed by a computer, cause the computer to perform the computer implemented method according to the first aspect.

### BRIEF DESCRIPTION OF THE DRAWINGS

FIG. 1 shows a typical mix design process for obtaining a mix proportion of an alkali activated concrete, AAC, mixture;

FIG. 2 shows steps according to a computer-implemented method for determining one or more physical properties of alkali activated concrete, AAC, from a mix proportion of an AAC mixture;

FIG. 3 shows steps for training a gradient boosted regression tree model for predicting the compressive strength of an AAC mixture according to an embodiment;

FIG. 4A shows example validation results of a gradient boosted regression tree model trained for determining the compressive strength of AAC based on control factors of the AAC mixture according to embodiments;

FIG. 4B shows example validation results of a random forest model trained for determining the compressive strength of AAC based on control factors of the AAC mixture according to embodiments;

FIG. 4C shows example validation results of an artificial neural network model trained for determining the compressive strength of AAC based on control factors of the AAC mixture according to embodiments;

FIG. 4D shows example validation results of a regression tree model trained for determining the compressive strength of AAC based on control factors of the AAC mixture according to embodiments;

FIG. 4E shows a comparison of the performance of different machine learning models for predicting the compressive strength of AAC that are trained and validated on the same training subset and testing subset;

FIG. 5 shows steps of the computer-implemented method for determining additional physical properties of the AAC from the mix proportion of the AAC mixture; and

FIG. 6 shows an example embodiment of a suitable computing system for performing steps according to example aspects of the invention.

### DETAILED DESCRIPTION OF EMBODIMENT(S)

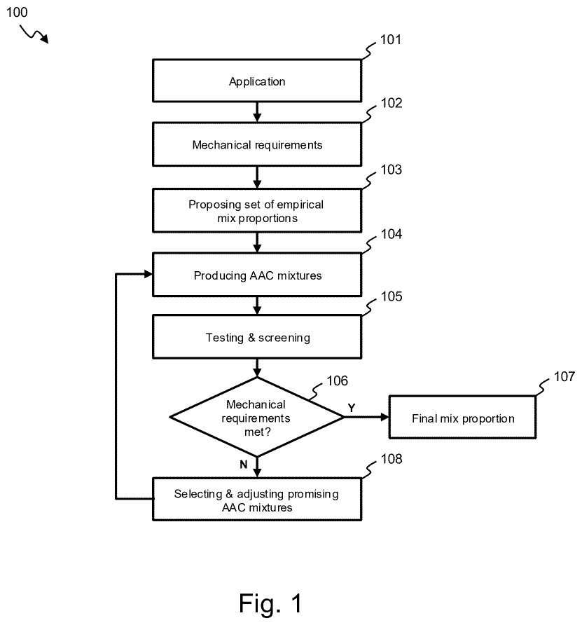

FIG. 1 shows a typical mix design process 100 for obtaining a mix proportion 107 of an alkali activated concrete, AAC, mixture. A mix proportion of an AAC mixture may be indicative of the ratio of constituents in the mixture in terms of volume or weight. Typically, the intended use or function of the hardened AAC, i.e. the application 101, determines the mechanical requirements 102 of the AAC, i.e. the desired physical properties. A set of empirical mix proportions is then typically proposed 103 based on these physical properties. These empirical mix proportions are typically proposed based on a limited set of mix proportions with known mechanical properties, based on limited guidance in literature, based on unreliable rules of thumb, based on expert experience, and/or based on conjecture. Typically, a plurality of empirical mix proportions is proposed in a trial-and-error manner, as it remains a problem to predict the resulting mechanical properties of the AAC.

In a following step 104, the proposed AAC mixtures are produced according to the proposed empirical mix proportions. This may comprise combining and mixing typical constituents of an AAC mixture such as, for example, precursors, an alkali activator, admixture, aggregates, and water. The plurality of AAC mixtures may then be tested and screened in step 105 to determine their respective mechanical properties. This testing is typically time and labour-intensive as the AAC mixtures have to set, harden, and/or cure sufficiently for some tests such as, for example, compression strength testing. As such, completing step 104 and step 105 typically takes between 28 and 90 days to complete.

In the most favourable case, one of the proposed AAC mixtures meets all the mechanical requirements, thereby obtaining the final mix proportion 107 in only one iteration. This mix proportion 107 may then serve as a recipe to produce substantial quantities of AAC mixture for the intended application 101. However, in the more common case, none of the initial proposed AAC mixtures meet all the mechanical requirements. As such, the most promising AAC mixtures may be selected and their respective mix proportions may be adjusted or altered in an attempt to approximate the desired mechanical properties. In other words, the mix proportion of some AAC mixtures may be changed slightly to propose a second generation of empirical AAC mixtures. As predicting the effect of the adjustments of the mix proportion on the resulting mechanical properties of the AAC remains a problem, steps 104, 105, and 106 may be repeated to produce, test, and screen the second generation of empirical AAC mixtures. This iteration may take up to another 90 days to complete, further contributing to the time and labour-intensiveness of designing AAC mixtures. Moreover, redesigning or adjusting the mix proportions does not guarantee that a suitable AAC mix is obtained, as the understanding of the effects of different design factors of the AAC mixture on the mechanical properties of AAC is limited.

As such, the widespread utilization of AAC remains limited even though AAC has promising application prospects as it can be at least equally performant and more ecological than traditional concrete, e.g. Portland cement concrete. It can thus be desirable to predict the physical properties of AAC based on a proposed mix proportion of an AAC mixture to avoid or limit the time and labour for experimentally testing the proposed mix proportions.

To this end, some models to predict physical properties of AAC have been developed. These models are typically limited to predicting a single physical property for a mix comprising specific constituent materials, a narrow range of mix proportions, and a fixed curing condition. Additionally, these models are typically based on a limited database of mix proportions and their corresponding experimentally determined physical properties. These models further typically consider correlated input variables which can lead to over-featuring or over-fitting of the prediction model. As such, the broad applicability of prediction models for predicting physical properties of varying AAC mixtures is a challenge.

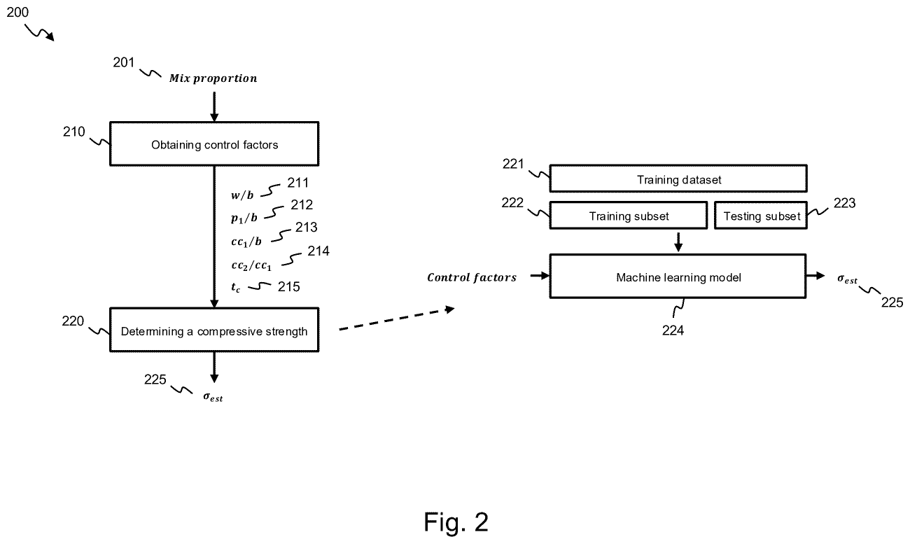

FIG. 2 shows steps 200 according to a computer-implemented method for determining one or more physical properties 225 of alkali activated concrete, AAC, from a mix proportion 201 of an AAC mixture. The AAC mixture may comprise one or more precursors, an alkali activator, aggregates, water, and/or admixtures. The one or more precursors, the alkali activator, and water may collectively form a cementitious material referred to as alkali activated paste, AAP. The alkali activator may be an aqueous solution of chemical compounds that enable the one or more precursors to react, thereby initiating the setting and hardening reactions of the AAC mixture, i.e. the solidifying the AAC mixture. The one or more precursors may be industrial by-products rich in alumina and/or silica.

The AAC mixture comprises a first precursor p₁, a second precursor p₂, a first chemical compound cc₁ of the alkali activator, and a second chemical compound cc₂ of the alkali activator. The first precursor p₁ may be blast furnace slag, BFS, the second precursor p₂ may be fly ash, FA, the first chemical compound cc₁ may be sodium oxide, Na₂O, and the second chemical compound cc₂ may be silicon dioxide, SiO₂, also referred to as silica. The alkali activator, comprising the first chemical compound cc₁ and the second chemical compound cc₂, may for example be obtained by mixing sodium hydroxide pearls, sodium silicate solution, and water. The first chemical compound of the alkali activator and/or the second chemical compound of the alkali activator may be an admixture or an additive. The aggregates included in the AAC mixture may be inert granular materials such as, for example, sand, gravel, or crushed stone. The admixtures may, for example, be air entrainers, water reducers, set retarders, set accelerators, superplasticizers, reactivity inhibitors, or any other admixture known to the skilled person.

In a first step 210, control factors 211-215 are obtained from the mix proportion 201 of the AAC mixture. It will be apparent that the control factors 211-215 may be obtained without producing the AAC mixture, i.e. without physically combining and mixing the constituents of the AAC mixture. The control factors 211-215 are indicative for one or more reaction mechanisms occurring during setting and/or hardening of the AAC mixture. In other words, the control factors 211-215 may be indicative for the reaction mechanisms that occur due to mixing the constituents of the AAC mixture according to the mix proportion. A control factor 211-215 may for example, amongst others, be indicative for the composition of reaction products, the structure of reaction products, a reaction rate of reactants, a dissolution rate of reactants, a condensation rate of reactants, or a saturation of reactants during or after hardening of the AAC mixture. As such, these control factors are characteristic for the physical properties of the obtained AAC.

The control factors 211-215 may thus be associated with the chemically active constituents in the AAC mixture, i.e. the precursors, the chemical compounds, and water. The control factors 211-215 may, for example, be relative amounts of the precursors, chemical compounds, water, or a combination thereof. This allows determining the compressive strength 225 of the AAC from the mix proportion 201 without considering the chemically inactive, i.e. inert, aggregates within the AAC mixture. This has the advantage that the compressive strength 225 can be determined or predicted for a wide variety of AAC mixtures, e.g. for AAC mixtures with different aggregates and varying mix proportions. In other words, the computer implemented method is broadly applicable to determine the compressive strength of AAC mixtures with substantially varying compositions.

The control factors 211-214 may be weight ratios indicative for a weight of one or more precursors, one or more chemical compounds, or water within the AAC mixture compared to a weight of the one or more precursors, the one or more chemical compounds, or the water within the AAC mixture. The control factors 211-214 may thus, for example, be a ratio between a weight of precursor and a total weight of precursor in the AAC mixture; a ratio between a weight of water and a weight of a chemical compound; a weight ratio between two chemical compounds; or any other weight ratio of chemically active constituents in the AAC mixture. This allows obtaining the control factors 211-214 from the mix proportion 201 of the AAC mixture in a straightforward manner. Alternatively, the control factors 211-214 may be volume ratios indicative for a volume of one or more precursors, one or more chemical compounds or water within the AAC mixture compared to a volume of the one or more precursors, the one or more chemical compounds, or the water within the AAC mixture.

The control factors 211-215 may further be a predetermined selection of factors that influence the compressive strength 225 of the AAC substantially through chemical reactions during the setting and hardening of the AAC mixture. In other words, a limited number of control factors 211-215 may be selected from a set of control factors that influence the compressive strength 225 of the AAC. The compressive strength 225 of the AAC may thus be characterized by a limited set of control factors 211-215. Such a limited number of control factors has the further advantage that over-featuring of the trained machine learning model 224 is avoided.

This predetermined selection of control factors 211-215 may comprise the weight ratio of water to the first and second precursor w/b 211, the weight ratio of the first precursor to the first and second precursor p₁/b 212, the weight ratio of the first chemical compound to the first and second precursor cc₁/b 213, and the weight ratio of the second chemical compound to the first chemical compound cc₂/cc₁ 214. The first and second precursor may collectively be referred to as the binder b. Weight ratio w/b 211 may be indicative of the pores that are introduced in the mixture; weight ratio p₁/b 212 may be indicative of the reaction capacity of the precursors, weight ratio cc₁/b 213 may be indicative of the intensity of the alkali-activated reaction; and weight ratio cc₂/cc₁ 214 may be indicative of the compactness of the structure.

In addition to relative amounts of chemically active constituents in the AAC mixture, determining the compressive strength of the AAC may further be based on a curing time 215 and/or a curing condition which may both influence the reaction mechanisms that occur during setting and/or hardening of the AAC mixture due to mixing the constituents of the AAC mixture according to the mix proportion 201.

The curing time 215 may be indicative for an elapsed time between the moment of initiating the hardening reaction of the AAC mixture, e.g. when placing or pouring the AAC mixture, and the moment when the compressive strength 225 is to be determined or predicted, e.g. 28 days after placing or pouring the AAC mixture. The curing time 215 may be expressed as a unit of time, e.g. hours or days. A curing condition may be indicative of the environmental conditions during the setting reactions of the AAC mixture, e.g. the curing condition may be ambient temperature or humidity.

In a following step 220, the compressive strength 225 of the AAC is determined based on the control factors 211-215 of the AAC mixture by means of a machine learning model 224. In other words, the control factors 211-215 associated with an AAC mixture may be provided to the machine learning model 224 as an input, which may then predict the compressive strength 225 of the resulting AAC as an output. The machine learning model 224 may be a gradient boosted regression tree, GBRT, model; a random forest, RF, model; an artificial neural network, ANN, model; or a regression tree, RT, model.

This has the advantage that the compressive strength 225 of an AAC mixture can be predicted in a cost-efficient manner, as the time and labour to determine the compressive strength of an AAC mixture can be reduced by avoiding curing and testing of AAC mixtures. It is a further advantage that the trained machine learning model 224 can be implemented in a mix design process for designing the mix proportion of an AAC mixture that yields AAC with desired physical properties. For example, the trained machine learning model 224 may be implemented in a typical mix design process as illustrated in FIG. 1, where the machine learning model 224 can be used in step 103 to propose a set of empirical mix proportions that have predicted physical properties close to the mechanical requirements 102 associated with the application 101. Thereby, mix designing of AAC mixture can be improved. It is a further advantage that this can improve the utilization of AAC as a more ecological alternative to traditional concrete.

The machine learning model 224 is trained on a training dataset 221 that includes control factors 211-215 associated with a plurality of AAC mixtures characterized by different mix proportions 201, and the corresponding compressive strengths of the AAC obtained by setting and hardening the plurality of AAC mixtures. The training dataset 221 may, for example, be a database of experimentally tested AAC mixtures wherein the control factors 211-215 of the AAC mixtures are mapped to the corresponding compressive strengths of the obtained AAC by setting and hardening of the respective AAC mixtures. For example, training data in the training dataset 221 may be structured as [(w/b, p₁/b, cc₁/b, cc₂/cc₁, t_{c}),(σexp)] wherein w/b, p₁/b, cc₁/b, cc₂/cc₁ and t_{c} are the control factors of an experimental AAC mixture; and wherein σₑₓₚ is the experimentally obtained compressive strength of the corresponding AAC obtained by curing a sample of the AAC mixture for a curing time t_{c}, e.g. 28 days. The compressive strength σₑₓₚ may, for example, be experimentally obtained by breaking an AAC sample in a compression-testing machine after a curing time t_{c}.

The training dataset 221 may comprise a substantial number of experimentally tested AAC mixtures, i.e. training data. In particular, the training dataset 221 may comprise at least around 800 experimentally tested AAC mixtures, preferably more than 800 experimentally tested AAC mixtures. Additionally, the training dataset 221 may further comprise experimentally tested AAC mixtures with substantially varying mix proportions, with substantially varying constituent materials, and/or with substantially varying curing conditions. The AAC mixtures in the training dataset 221 may further be compression tested after substantially different curing times. For example, the training dataset 221 may comprise 871 experimentally tested AAC mixtures characterized by a weight ratio w/b 211 between around 0.2 and around 0.8; a weight ratio p₁/b 212 between around 0 and around 1; a weight ratio cc₁/b 213 between around 1.3% and around 14.5%; a weight ratio cc₂/cc₁ 214 between around 0 and around 2.65; a curing time t_{c} 215 between around 1 day and around 180 days; and an experimentally tested compressive strength σₑₓₚ between around 0.30 MPa and around 120 MPa.

The substantial number of AAC mixtures in the training dataset 221 and their varying characteristics has the further advantage that the trained machine learning model 224 can be more accurate and can be more broadly applicable to determine the compressive strength 225 of AAC mixtures with varying compositions. It is a further advantage that the compressive strength 225 can be determined for AAC mixtures comprising a plurality of precursors and/or a plurality of chemical compounds.

The training dataset 221 may further be divided into a training subset 222 for training the machine learning model 224 and a testing subset 223 for validating the trained machine learning model 224. The performance of a trained machine learning model 224 may be evaluated by one or more validation metrics, e.g. a coefficient of determination, a mean absolute percentage error, MAPE, a root mean square error, RMSE, and a mean absolute error, MAE. These validation metrics may respectively be determined as

$${R}^{2} = 1 - \frac{{\sum }_{i = 1}^{N} ( {σ}_{est , i} - {σ}_{ex p , i} )}{{\sum }_{i = 1}^{N} {( {σ}_{est , i} - {\overline{σ}}_{e x p} )}^{2}} \qquad ( Eq . 1 )$$

$$MAPE = \frac{100 \%}{N} {\sum }_{i = 1}^{N} \frac{\vert {σ}_{est , i} - {σ}_{ex p , i} \vert}{{σ}_{e xp , i}} \qquad ( Eq . 2 )$$

$$RMSE = \sqrt{\frac{{\sum }_{i = 1}^{N} {( {σ}_{est , i} - {σ}_{e xp , i} )}^{2}}{N}} \qquad ( Eq . 3 )$$

$$MAE = \frac{{\sum }_{i = 1}^{N} \vert ( {σ}_{est , i} - {σ}_{e xp , i} ) \vert}{N} \qquad ( Eq . 4 )$$

wherein N is the number of experimentally tested AAC mixtures in the testing subset 223.

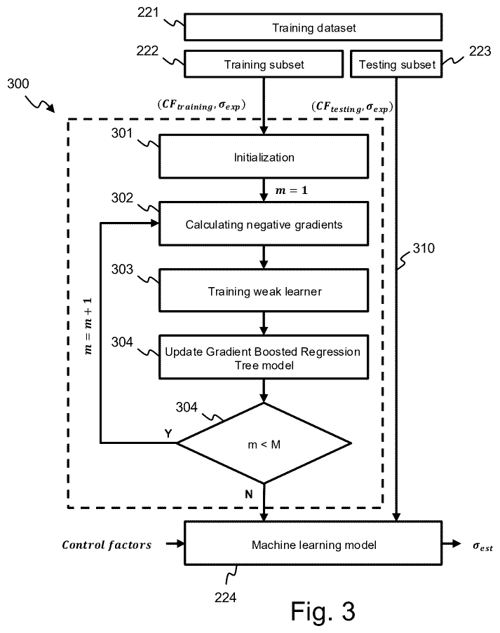

FIG. 3 shows steps 300 for training a gradient boosted regression tree, GBRT, model for predicting the compressive strength of an AAC mixture according to an embodiment. The training is based on the training subset 222 of the training dataset 221, which comprises training data (CF_{training}, σₑₓₚ) as described above in relation to FIG. 2. In a first step 301, the GBRT may be initialized, e.g. by setting F₀(CF_{training})=mean(σₑₓₚ). In a following step 302, negative gradients g; may be determined for each training sample (CF_{training,i}, σ_{exp,i}) as, for example

$${g}_{i} = - {[ \frac{δ l ( {σ}_{ex p , i} , {F}_{m - 1} ( C {F}_{training , i} ) )}{δ {F}_{m - 1} ( C {F}_{training , i} )} ]}_{F = {F}_{m - 1}} \qquad ( Eq . 5 )$$

wherein δ is a derivative and l is a loss function.

In a next step 303, the m^{th} weak learner ŷ_{k,m} may be trained by

$${\hat{y}}_{k , m} = arg min {\sum }_{C {F}_{training , i} ∈ {R}_{i , m}} l ( {σ}_{e xp , i} , {F}_{m - 1} ( {CF}_{training , i} ) + {\hat{y}}_{k , m} ) \qquad ( Eq . 6 )$$

wherein R_{i,m} represents the leaf nodes of the gradient boosted regression tree. In the following step 304, the GBRT model is updated as

$${F}_{m} ( C {F}_{training} ) = {F}_{m - 1} ( C {F}_{training} ) + v {\sum }_{k = 1}^{K} {\hat{y}}_{k , m} I ( {CF}_{training} ∈ {R}_{k , m} ) \qquad ( Eq . 7 )$$

wherein K represents the number of lead nodes, v is the learning rate, and I represents an indicator function. If counter m is smaller than a predetermined value M in step 304, counter m may be incremented by one and steps 302, 303, and 304 may be iterated until counter m equals predetermined value M. The obtained GBRT model, i.e. the trained machine learning model 224, may then for example be represented as

$${F}_{M} ( C {F}_{training} ) = {\sum }_{m = 1}^{M} {\sum }_{k = 1}^{K} v {\hat{y}}_{k , m} I ( {CF}_{training} ∈ {R}_{k , m} ) \qquad ( Eq . 8 )$$

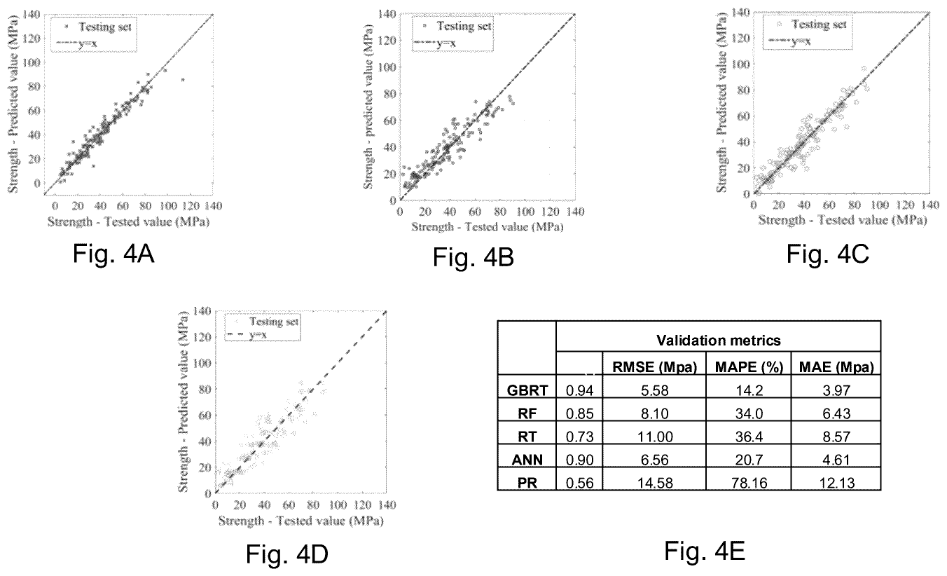

Hereafter, the trained GBRT model 224 may be validated 310 based on the testing subset 223 of the training dataset 221. FIG. 4A shows example validation results of such a GBRT model trained for determining the compressive strength of AAC based on control factors of the AAC mixture according to embodiments. The GBRT model is trained based on the above mentioned example training dataset of 871 experimentally tested AAC mixtures. The trained GBRT model allows predicting the compressive strength of the AAC with an R² value of at least 0.94, an RMSE value of 5.58 MPa, a MAPE value of 14.2%, and a MAE value of 3.97 MPa.

FIG. 4B shows example validation results of a RF model trained for determining the compressive strength of AAC based on control factors of the AAC mixture according to embodiments. The RF model is trained and validated on the same example training dataset as the RF model of FIG. 4A. The trained RF model allows predicting the compressive strength of the AAC with an R² value of at least 0.85, an RMSE value of 8.10 MPa, a MAPE value of 34%, and a MAE value of 6.43 MPa.

FIG. 4C shows example validation results of an ANN model trained for determining the compressive strength of AAC based on control factors of the AAC mixture according to embodiments. The ANN model is trained and validated on the same example training dataset as the GBRT model of FIG. 4A and the RF model of FIG. 4B. The trained ANN model allows predicting the compressive strength of the AAC with an R² value of at least 0.9, an RMSE value of 6.56 MPa, a MAPE value of 20.7%, and a MAE value of 4.61 MPa.

FIG. 4D shows example validation results of a RT model trained for determining the compressive strength of AAC based on control factors of the AAC mixture according to embodiments. The RT model is trained and validated on the same example training dataset as the GBRT model, RF model, and ANN model of FIG. 4A, FIG. 4B, and FIG. 4C, respectively. The trained RT model allows predicting the compressive strength of the AAC with an R² value of at least 0.73, an RMSE value of 11.00 MPa, a MAPE value of 36.4%, and a MAE value of 8.57 MPa.

FIG. 4E shows a comparison of the performance of the different machine learning models that are trained and validated on the same training subset and testing subset, respectively. FIG. 4E further shows the performance of a polynomial regression, PR. It will be apparent from FIG. 4E that the proposed machine learning models for predicting the compressive strength are more accurate compared to a simple polynomial fit of the training subset. The GBRT model allows predicting the compressive strength of AAC more accurately, more efficiently, and faster compared to the ANN model, the RF model and the RT model.

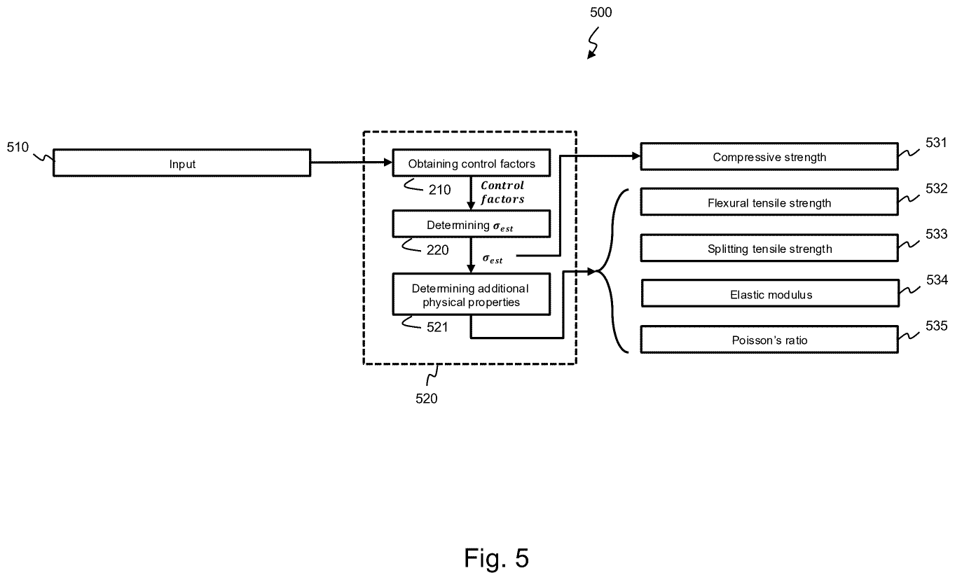

FIG. 5 shows steps 520 of the computer-implemented method for determining additional physical properties 532-535 of the AAC from the mix proportion of the AAC mixture. In a first step 210, the control factors may be obtained from an input 510 as discussed above in relation to FIG. 2. The input 510 may be the complete mix proportion of the AAC mixture, e.g. the respective masses of all constituents in the AAC mixture. Alternatively, the input 510 may be a portion of the mix proportion, e.g. only the mass of the one or more precursors in the AAC mixture, the mass of the one or more chemical compounds in the AAC mixture, and the mass of water in the AAC mixture. The input 510 may further include, amongst others, the state of the chemical compounds used to prepare the alkali activator, and/or the concentration of the one or more chemical compounds in starting solutions or starting products used to prepare the alkali activator. Alternatively or complementary, the input 510 may include the curing time of the AAC, one or more curing conditions of the AAC, and/or one or more of the control factors, e.g. the weight ratio w/b, the weight ratio p₁/b, the weight ratio cc₁/b, the weight ratio cc₂/cc₁.

In a next step 220, the trained machine learning model may predict the compressive strength σₑₛₜ of the AAC based on the obtained control factors, as described above in relation to FIG. 2. The predicted compressive strength σₑₛₜ may be a first output 531 of the computer-implemented method 500.

In a following step 521, one or more additional physical properties 532-535 of the AAC may be determined from the predicted compressive strength σₑₛₜ. The additional physical properties 532-535 may include the flexural tensile strength 532 of the AAC, the splitting tensile strength 533 of the AAC, the elastic modulus 534 of the AAC, and/or the Poisson's ratio 535 of the AAC. The additional physical properties 532-535 may be determined based on respective predetermined relationships with the compressive strength 531. The predetermined relationships may, for example, be mathematical functions that map the compressive strength of AAC to the respective additional physical properties 532-535. In other words, the determined compressive strength value σₑₛₜ may be an input variable for the respective predetermined relationships, which then output the predicted flexural strength 532, splitting tensile strength 533, elastic modules 534, and/or Poisson's ratio 535 of the AAC.

This allows predicting a plurality of physical properties 531-535 of AAC from the mix proportion of the AAC mixture, i.e. from the input 510. This has the further advantage that the time and labour to determine physical properties of an AAC mixture can further be reduced by further avoiding curing and testing of AAC mixtures. The respective predetermined relationships may, for example, be determined by a polynomial fitting on a dataset comprising the physical properties 531-535 of a plurality of AAC mixtures characterized by different mix proportions.

The predetermined relationship between the determined or predicted compressive strength σₑₛₜ 531 and the flexural strength fᵣ 532 may be a square root relationship, such as, for example:

$${f}_{r} = 0.83 \sqrt{{σ}_{est}} \qquad ( Eq . 9 )$$

The predetermined relationship between the determined or predicted compressive strength σₑₛₜ 531 and the splitting tensile strength fₛₚ 533 may be a square root relationship, such as, for example:

$${f}_{s p} = 0.58 \sqrt{{σ}_{est}} \qquad ( Eq . 10 )$$

The predetermined relationship between the determined or predicted compressive strength σₑₛₜ 531 and the elastic modulus fₛₚ 534 may be a square root relationship, such as, for example:

$${f}_{s p} = 4250 \sqrt{{σ}_{est}} \qquad ( Eq . 11 )$$

These predetermined relationships have the further advantage that the additional physical properties 532-535 may be determined fast and computationally efficient, as the computations for determining the flexural strength 532, splitting tensile strength 533, and elastic modulus 534 require limited computing resources.

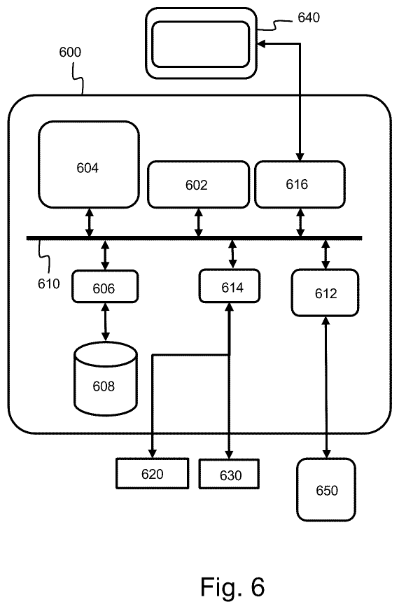

FIG. 6 shows a suitable computing system 600 enabling to implement embodiments of the above described method according to the invention. Computing system 600 may in general be formed as a suitable general-purpose computer and comprise a bus 610, a processor 602, a local memory 604, one or more optional input interfaces 614, one or more optional output interfaces 616, a communication interface 612, a storage element interface 606, and one or more storage elements 608. Bus 610 may comprise one or more conductors that permit communication among the components of the computing system 600. Processor 602 may include any type of conventional processor or microprocessor that interprets and executes programming instructions. Local memory 604 may include a random-access memory (RAM) or another type of dynamic storage device that stores information and instructions for execution by processor 602 and/or a read only memory (ROM) or another type of static storage device that stores static information and instructions for use by processor 602. Input interface 614 may comprise one or more conventional mechanisms that permit an operator or user to input information to the computing device 600, such as a keyboard 620, a mouse 630, a pen, voice recognition and/or biometric mechanisms, a camera, etc. Output interface 616 may comprise one or more conventional mechanisms that output information to the operator or user, such as a display 640, etc. Communication interface 612 may comprise any transceiver-like mechanism such as for example one or more Ethernet interfaces that enables computing system 600 to communicate with other devices and/or systems such as for example, amongst others, a system for mixing an AAC mixture 650. The communication interface 612 of computing system 600 may be connected to such another computing system by means of a local area network (LAN) or a wide area network (WAN) such as for example the internet. Storage element interface 606 may comprise a storage interface such as for example a Serial Advanced Technology Attachment (SATA) interface or a Small Computer System Interface (SCSI) for connecting bus 610 to one or more storage elements 608, such as one or more local disks, for example SATA disk drives, and control the reading and writing of data to and/or from these storage elements 608. Although the storage element(s) 608 above is/are described as a local disk, in general any other suitable computer-readable media such as a removable magnetic disk, optical storage media such as a CD or DVD, -ROM disk, solid state drives, flash memory cards, etc. could be used.

Although the present invention has been illustrated by reference to specific embodiments, it will be apparent to those skilled in the art that the invention is not limited to the details of the foregoing illustrative embodiments, and that the present invention may be embodied with various changes and modifications without departing from the scope thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the invention are capable of operating according to the present invention in other sequences, or in orientations different from the one(s) described or illustrated above.

1. A computer implemented method for determining one or more physical properties of alkali activated concrete, AAC, from a mix proportion of an AAC mixture; wherein the AAC mixture comprises a first chemical compound of an alkali activator, a second chemical compound of the alkali activator, a first precursor, a second precursor, and water according to the mix proportion; the computer implemented method comprising:
obtaining control factors from the mix proportion of the AAC mixture, wherein the control factors are weight ratios including a weight ratio of water to the first and second precursor, a weight ratio of the first precursor to the first and second precursor, a weight ratio of the first chemical compound to the first and second precursor, and a weight ratio of the second chemical compound to the first chemical compound; and
determining a compressive strength of the AAC based on the control factors of the AAC mixture by means of a machine learning model;
wherein the machine learning model is trained by a training dataset comprising control factors of a plurality of AAC mixtures with different mix proportions; and corresponding compressive strengths of the AAC obtained by setting and hardening of the respective AAC mixtures.

2. The computer implemented method according to claim 1, wherein the first chemical compound is sodium oxide, Na₂O, the second chemical compound is silicon oxide, SiO₂, the first precursor is blast furnace slag, BFS, and the second precursor is fly ash, FA.

3. The computer implemented method according to claim 1, wherein determining the compressive strength of the AAC is further based on a curing time and/or a curing condition of the AAC mixture.

4. The computer implemented method according to claim 3, wherein the curing time of the AAC mixture is between about 1 day and about 180 days.

5. The computer implemented method according to claim 1, wherein the machine learning model is a gradient boosted regression tree, GBRT, model, a random forest, RF, model, an artificial neural network, ANN, model, or a regression tree, RT, model.

6. The computer implemented method according to claim 1, further comprising determining a flexural strength, a splitting tensile strength, and/or an elastic modulus of the AAC based on respective predetermined relationships with the determined compressive strength.

7. The computer implemented method according to claim 6, wherein the respective predetermined relationships are square root relationships between the determined compressive strength and the flexural strength, the determined compressive strength and the splitting tensile strength, and/or the determined compressive strength and the elastic modules.

8. A data processing system configured to perform the computer implemented method according to claim 1.

9. A computer program comprising instructions which, when the computer program is executed by a computer, cause the computer to perform the computer implemented method according to claim 1.

10. A computer-readable medium comprising instructions which, when executed by a computer, cause the computer to perform the computer implemented method according to claim 1.
